(19) United States
(12) Patent Application Publication
(10) Pub. No.: US 2012/0107293 A1
(43) Pub. Date: May 3, 2012

(54) METHODS AND COMPOSITIONS FOR THE TREATMENT OF CANCER

(76) Inventors: Zheng Cui, Winston-Salem, NC (US); Mark C. Willingham, Winston-Salem, NC (US)

(21) Appl. No.: 13/282,832

(22) Filed: Oct. 27, 2011

Related U.S. Application Data
(62) Division of application No. 11/870,768, filed on Oct. 11, 2007, now abandoned.
(60) Provisional application No. 60/829,416, filed on Oct. 13, 2006.

(51) Int. Cl. A61K 35/14; C40B 30/06; A61P 35/00; C12Q 1/02

(52) U.S. Cl. 424/93.71; 435/29; 506/10

(57) ABSTRACT

A method of treating cancer comprises: (a) providing allogenic or autologous white blood cells from a suitable donor; and then (b) administering the white blood cells to the subject in an amount effective to treat the cancer. Preferably the white blood cells comprise innate immune cells. Preferably the white blood cells comprise less than 10% by number of cytotoxic T lymphocytes. Preferably the white blood cells, or more particularly the innate immune cells, are preselected in vitro to kill cancer cells in vitro (for example, by collecting white blood cells from the patient and determining that the white blood cells kill cancer cells in vitro before and thereby pre-selecting the donor, before collecting a subsequent population of cells from the donor for administration).

### RELATED APPLICATIONS

This application claims the benefit of U.S. Provisional Patent Application Ser. No. 60/829,416, filed Oct. 13, 2006, the disclosure of which is incorporated by reference herein in its entirety.

### FIELD OF THE INVENTION

The present invention concerns methods and compositions useful for the treatment of cancer by techniques related to adoptive immunotherapy.

### BACKGROUND OF THE INVENTION

Cancer is a devastating disease in humans, as well as veterinary subjects such as dogs and cats. For example, 25% of humans and 50% of pet dogs die of cancer. Current therapies include surgery, radiation and cytotoxic chemotherapies. Many of these are ultimately ineffective, and accompanied by harmful side-effects. Leukocyte infusions have been employed to treat human cancer (Schwarzenberg, et al. (1966) Lancet 2(7459):365-8; Porter, et al. (1999) J. Clin. Oncol. 17(4):1234; Strair, et al. (2003) J. Clin. Oncol. 21(20):3785-91), wherein the response of the cancer patients was proportional to the number of leukocytes received (Schwarzenberg, et al. (1966) Lancet 2(7459):365-8).

The age-adjusted cancer death rate in the U.S. (about 200 in per 100,000 people in the general population) has not changed since the 1950s when post-war cancer mortality data collection first resumed. On the other hand, 75% of humans do not die of cancer, and even most cancer patients remain cancer-free for most of their lifespan. Indeed, some humans remain cancer-free into their 80s and 90s even with daily-exposures to known carcinogens, such as heavy cigarette smoking. However, the molecular basis for why these individuals do not get cancer has not been determined.

### SUMMARY OF THE INVENTION

A first aspect of the invention is a method of treating cancer in a subject in need thereof, comprising: (a) providing allogenic white blood cells from a suitable donor; and then (b) administering the white blood cells to the subject in an amount effective to treat the cancer. Preferably the white blood cells comprise, consist essentially of or consist of innate immune cells. Preferably the white blood cells, or more particularly the innate immune cells, are preselected in vitro to kill cancer cells in vitro (for example, by collecting white blood cells from the patient and determining that the white blood cells kill cancer cells in vitro before and thereby pre-selecting the donor, before collecting a subsequent population of cells from the donor for administration).

A second aspect of the invention is a pharmaceutical formulation comprising, consisting of or consisting essentially of white blood cells (e.g., innate immune cells) as described herein in a pharmaceutically acceptable carrier.

A still further aspect of the present invention is the use of white blood cells (e.g., innate immune cells) as described herein for the preparation of a medicament for the treatment of cancer.

A still further aspect of the invention is a method of screening innate immune cells in vitro for cancer killing activity, comprising: providing white blood cells comprising innate immune cells; then contacting then white blood cells to cancer cells in vitro for a period of time; and then detecting whether or not the innate immune cells kill the cancer cells. White blood cells such as innate immune cells that kill the cancer cells in vitro are useful in the in vivo methods of treatment described herein.

The present invention is explained in greater detail in the specification set forth below. The disclosures of all US patent references cited herein are to be incorporated by reference herein in their entirety.

### BRIEF DESCRIPTION OF THE DRAWINGS

FIG. 1. CKA by Age and Health Status. Wild-type (WT) and spontaneous regression (SR) mouse samples were included for comparison. Horizontal bars indicate geometric mean of CKA distribution within a given sample. Y denotes years.

FIG. 2. Representative Samples of CKA Development. Arrows indicate addition of effectors to non-control groups. FIG. 2A shows CKA results using RT-CES output. X denotes results of the control, whereas Y denotes cell death and decreased adherence mediated by effector cell populations of three separate individuals. FIG. 2B is a graph of CKA using various effector-to-target cell ratios. FIG. 2C illustrates granulocyte and agranulocyte effector functionality. FIG. 2D shows the seasonal phenomenon of CKA among three individuals.

### DETAILED DESCRIPTION OF THE PREFERRED EMBODIMENTS

“White blood cell” or “leukocyte” as used herein refers to any type of white blood cell, including adaptive immune cells and innate immune cells.

“Adaptive immune cells” (or “memory immune cells”) as used herein has its conventional meaning and includes T-cells and B-cells.

“Innate immune cells” as used herein has its conventional meaning and includes polymorphonuclear leukocytes (i.e., granulocytes, such as neutrophils, basophils and eosinophils), monocyte/macrophages (depending upon their source of collection), and natural killer cells.

“Allogenic” as used herein refers to blood or blood cells from a donor that is different from the recipient (though typically of the same species). Where the donor and the recipient are the same, the blood or blood cells are “autologous”.

“Subjects” as used herein are generally mammalian subjects, particularly including human subjects, and veterinary subjects such as dogs, cats, horses, sheep, goats, and primates such as monkeys and chimpanzees. Subjects may be of any age including infant, child or pre-adolescent, adolescent, adult, or geriatric subjects.

“Cancer” as used herein may be any cancer, including but not limited to lung, colon, liver, prostate, ovarian, breast, brain, thyroid, bone, kidney and skin (e.g., melanoma) cancers, as well as cancers such as leukemia and lymphoma.

### A. Donors and Cell Selection.

Donors of white blood cells, also referred to herein as effector cells, used to carry out the present invention are preferably identified by a preselection process in which a sample of white blood cells are collected from the donor and screened in vitro for the ability to kill cancer cells in vitro. Thus, the donors are typically healthy allogenic donors. In some embodiment the donor is autologous: that is, the same subject as being treated, but having donated the cells at an earlier point in time prior to the development of disease. Any suitable cancer cells or target cells can be used, including but not limited to S180 cells. The white blood cells can be contacted to a plurality (or “panel”) of cancer cells (e.g., 2, 4, 6, or 8 or more different cancer cells) to identify cells effective against a variety of diseases. The cancer cells can be from the same species or a different species as the subject being treated, and the white blood cells can be screened against a single cancer cell line or multiple cell lines. Indeed, the white blood cells can be screened in vitro against cancer cells collected from the subject to whom the cells are ultimately administered.

Any suitable screening assay format can be employed. In general, the method may be carried out by first providing white blood cells (e.g., innate immune cells) collected from the donor (e.g., a human or dog donor). The white blood cells are then contacted to cancer cells in vitro for a period of time (e.g., from 6 hours, 12 hours, or 1 day up to 3 or 6 days). The contacting step is preferably carried out at a temperature greater than room temperature (e.g., of from 35° C. or 36° C. up to 41° C. or 42° C.). Whether or not cancer cells have been killed (in whole or in significant numbers) by the white blood cells can then be detected by any of a variety of techniques, including but not limited to phase contrast microscopy and/or fluorescence microscopy. In one preferred embodiment, the detecting step is carried out by cell electronic sensing, such as with the RT-CES™ system available from ACEA Biosciences, Inc. (11585 Sorrento Valley Rd., Suite 103, San Diego, Calif. 92121 USA). The cancer cells may be any suitable cancer cells, optionally from the same species as the white blood cell donor, examples including but not limited to lung, colon, liver, prostate, ovarian, breast, brain, kidney, skin, leukemia and lymphoma cancer cells. In one embodiment, the white blood cells are screened against a plurality of different cancer cells (e.g., different ones of the aforesaid types of cancer cells), so that cells of particular efficacy for killing a particular cancer can then be identified.

Optionally, but in some embodiments preferably, the donor may be administered a white blood cell growth factor in accordance with known techniques prior to white blood cell collection. Suitable growth factors include but are not limited to granulocyte-macrophage colony-stimulating factor (GM-CSF), Interleukin-4 (IL-4), Interleukin-6 (IL-6), TNF-alpha, granulocyte colony-stimulating factor (G-CSF), macrophage colony-stimulating factor (M—CSF), and Interleukin-18 (IL-18). See, e.g., U.S. Pat. No. 6,893,633. Particular examples of the foregoing include, but are not limited to, LEUKINE® brand sargramostim, NEUPOGEN® brand filgrastim, and NEULASTA® brand PEG-filgrastim.

Once a suitable donor is identified, additional cells can be collected from that donor by any suitable technique, including but not limited to bone marrow aspiration, spleen cell harvesting, from peripheral blood, e.g., by leukopheresis in accordance with known techniques (see, e.g., U.S. Pat. Nos. 4,111,199 and 4,690,915). Alternatively, cells collected from a donor for other reasons can be screened for in vitro cancer killing activity as described herein and then used for the methods described herein.

White blood cells can optionally be sorted into particular subcategories or types in accordance with any suitable technique. Such sorting includes separation of granulocytes (e.g., neutrophils, basophils and eosinophils) from agranulocytes (e.g., lymphocytes, monocytes and macrophages). In one embodiment, the white blood cells are sorted by counter-flow centrifugal elutriation, such as with the ELUTRA™ cell separation system available from Gambro BCT (10810 West Collins Avenue, Lakewood, Colo. 80215 USA).

White blood cells collected, and optionally sorted, can be grown or expanded by in vitro culture before administration to the recipient subject in accordance with known techniques, including but not limited to those described in U.S. Pat. Nos. 5,541,105 and 4,690,915. Culture media may optionally include one or more of the growth factors described above. When grown as particular subtypes, the white blood cells can optionally be recombined to produce the desired composition for administration.

Given that CKA can be suppressed during the winter season, stress, aging and because of inferior genetics, white blood cells of the invention can be obtained, e.g., during the summer or from young, healthy donors and stored for subsequent use in the treatment of cancer.

### B. Preparation and Administration.

The present invention can be carried out in accordance with techniques known to those skilled in the art (see, e.g., U.S. Pat. Nos. 6,770,749; 6,322,790; 6,156,302; 5,776,451; 5,229,115; 5,081,029; and 4,690,915), as modified in light of the disclosure provided herein.

In general, the white blood cells used to carry out the invention are combined with a pharmaceutically acceptable carrier (e.g., an injectible carrier such as sterile physiological saline solution). The formulation can be prepared in unit dosage faun (e.g., in a vial or ampoule for injection) containing the appropriate number of cells for administration in a single dose, or split among two, three or more doses, as discussed below.

Leukocytes or white blood cells used for administration to the recipient can be tissue-matched to the recipient or selected to be histocompatible with the recipient subject, in accordance with known techniques. See, e.g., U.S. Pat. Nos. 5,776,588; 5,032,407; and 4,921,667. However, in some embodiments, the white blood cells are preferably not tissue matched and not histocompatible with the recipient subject, so that the white blood cells are ultimately rejected in whole or in part by the recipient.

The white blood cells can be sorted or enriched for particular subpopulations for administration, as noted above. For example, in some embodiments the white blood cells administered contain less than 30%, less than 20%, less than 10%, less than 5%, or less than 1% by number of adaptive immune cells (or in a particular embodiment, less than 30%, less than 20%, less than 10%, less than 5%, or less than 1% by number of cytotoxic T lymphocytes). In some embodiments, the white blood cells administered comprise, consist of, or consist essentially of innate immune cells. Thus, in some embodiments the white blood cells are free of, or essentially free of, adaptive immune cells. Reduction or substantial exclusion of adaptive immune cells may be advantageous in some embodiments, such as where the white blood cells are administered to an immune compromised patient.

Similarly, in some embodiments (such as for administration to an immune compromised patient) it may be advantageous to irradiate the white blood cells with a suitable dose of ionizing radiation (e.g., with from 5 or 10 to 40 or 50 gray, preferably 20 to 30 gray, most preferably 25 gray) to reduce the proliferative capacity thereof.

Administration can be by any suitable technique or route, including but not limited to intraveneous injection (e.g., into a major peripheral vein), intraarterial injection, (e.g., into the hepatic artery), intraperitoneal injection, injection into a tumor resection cavity, intrathecal injection, etc. The amount of white blood cells administered can be determined in accordance with known techniques depending upon the size and condition of the subject, the route of administration, the particular formulation administered, etc., but in general may be from 10⁶, 10⁷, 10⁸ or 10⁹ cells, up to 10¹², 10¹³ or 10¹⁴ of the white blood cells or more. Administration may be carried out once, or repeated one, two or three or more times as necessary.

Optionally, but in some embodiments preferably, the subject may be administered a white blood cell growth factor concurrently with (including just prior to) or after administration of the white blood cells. Suitable white blood cell growth factors include but are not limited to granulocyte-macrophage colony-stimulating factor (GM-CSF), Interleukin-4 (IL-4), Interleukin-6 (IL-6), TNF-alpha, granulocyte colony-stimulating factor (G-CSF), macrophage colony-stimulating factor (M-CSF), and Interleukin-18 (IL-18). See, e.g., U.S. Pat. No. 6,893,633. Particular examples of the foregoing include but are not limited to LEUKINE® brand sargramostim, NEUPOGEN® brand filgrastim, and NEULASTA® brand PEG-filgrastim.

Treatment of a subject with the preselected white blood cells of the invention desirably achieves at least a 30% decrease in the sum of the longest diameter (LD) of target lesions taking as reference the baseline sum LD; a complete response, wherein all lesions disappear and tumor marker level is normalized; or stabilization of lesion growth such there is no significant increase in the size of the lesion, taking as references the smallest sum LD since the treatment started. Lesions can be monitored by conventional methods such as cytology or histology.

The present invention is explained in greater detail in the following non-limiting Examples.

### Example 1

### Cancer-Cell-Killing Activity of White Blood Cells

SR/CR (spontaneous regression/complete resistance) mice are a colony of unique cancer-resistant mice developed from a single male mouse that unexpectedly survived challenges with lethal cancer cells (Cui et al. (2003) Proc. Natl. Acad. Sci. USA 100:6682-6687). This highly effective natural cancer immunity or resistance is determined by inheritance, and is mediated entirely by white blood cells (WBCs). This resistance is exceptionally effective against a wide array of lethal transplantable or endogenous malignancies in mice. More importantly, this immunity can be transferred via WBCs from cancer-resistant mice to ordinary mice for highly effective cancer treatment and cancer prevention. Indeed, when wild-type mice with lethal prostate cancer induced by prostate-specific knockout of PTEN gene were treated with leukocytes transfused from SR/CR mice, 100% of treated mice were cured. The lifespan of the treated mice doubled from 7 months to 14 month and the entire prostates became scar tissues, indicating that these leukocytes from the SR/CR mice had anticancer properties. Moreover, unlike any current cancer therapies, this cancer resistance, endogenous or transferred, is not associated with any adverse side-effects. These findings in mice have laid a conceptual framework for adoptively transferring WBCs from cancer-resistant individuals to cancer patients for treatment and prevention of cancers. However, identification of cancer-resistant humans as WBC donors is required.

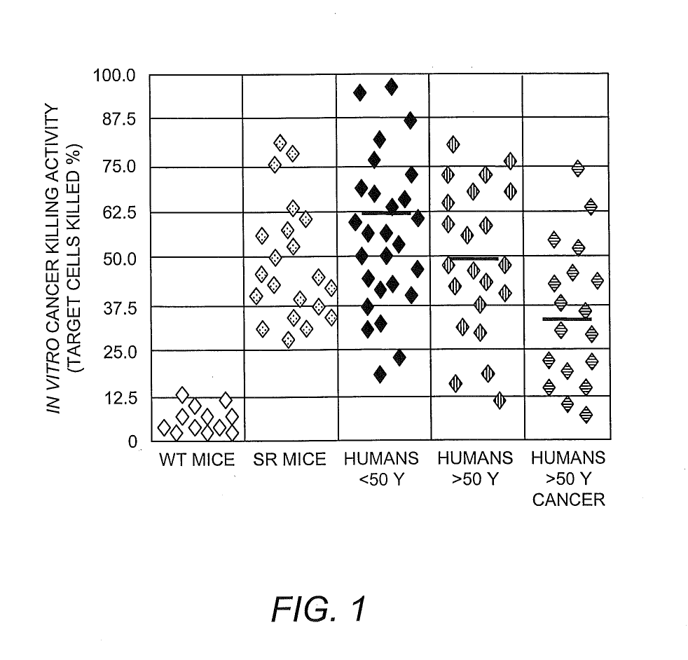

Cancer-resistant mice can be easily identified by their survival after challenge with lethal transplantable cancer cells. Ordinary mice uniformly die with the same challenge. The cancer-resistant mice can also be identified and distinguished by measuring the ability of WBCs for killing cancer cells in test tubes (in vitro) without having to challenge mice with live lethal cancer cells. Cancer-resistant mice have high cancer-killing activity and ordinary mice have no activity. Using its highly accurate predictability of cancer resistance for mice, the in vitro assay was adapted into a human blood test. After sampling a group of volunteers using this unique blood test, it was found that healthy humans had a wide range of cancer-cell-killing activity (CKA; FIG. 1). On a 0% to 100% scale, WBCs from many healthy humans had significant levels of naturally-present activity ranging between 40% and 60%, with some as high as that of cancer-resistant mice at levels of 70% to 90%. Activities in some individuals and cancer patients were significantly low, similar to that of ordinary mice at 0% to 20%. Overall, healthy persons had a higher CKA than their age-matched counterparts whom had cancer. Intriguingly, individuals over the age of 50 also demonstrated an overall lower CKA than a younger comparison group. Similar to the trend in mice, the CKA trend among humans is reflective of the sample's cancer status. Therefore, this analysis indicates that, as in mice, this highly accurate blood test can be used to predict anti-cancer status in humans. Furthermore, the WBCs in the people with exceptional activity (at 70-90% level or better) may have therapeutic effect when adoptively transferred to cancer patients. Moreover, healthy people with average activities can be boosted with a unique method to become donors with exceptional activity against general cancers or a specific cancer. Because leukocyte transfusion is practiced safely on a regular basis in hospitals and there is no involvement of new synthetic compounds, the instant treatment strategies can be readily implemented in humans.

### Example 2

### In Vitro Cancer-Killing-Activity Assay

Human and dog populations contain various levels of cancer-killing-activity (CKA) in their white blood cells (WBC), especially their innate immune system WBCs. Thus, cells of use in accordance with the present invention are first preselected in vitro for their ability to kill cancer cells. It is contemplated that one cell type or a panel of different cells can be employed in this in vitro assay to accurately predict the anti-cancer activity of WBCs.

Target cells, e.g., HeLa cells, were prepared according to the following protocol. Cells were cultured in DMEM+10% FBS (fetal bovine serum) in a T25 flask to 80% confluence. Cells were trypsinized, harvested and counted with Trypan Blue. Assay plates (24-well) were seeded with 1.5×10⁴ cells per well in 24-well flat bottom plates. Plates were incubated at 37° C. in 5% CO₂ for 24 hours. Cells were labeled with 2.5 μM CellTracker™ Green for 45 minutes. Fresh medium was added to cells and they were placed back into a CO₂-incubator.

WBCs were collected by drawing approximately 18 ml of human blood from a subject. The blood was split into three BD Vacutainer™ CPT tubes and centrifuged at 175×g for 35 minutes at 23° C. The mononuclear cell (MN) layer was collected and transferred to a 15 ml conical tube. The MN cells were centrifuged at 420×g for 5 minutes at 23° C. and washed with 10 ml DMED+10% FBS. Cells were counted and resuspended in medium to a final concentration of 1.6×10⁶ cells/ml.

The CKA assay was carried out by adding 500 μl of MN cell suspension (8×10⁵ cells total) to each well in which HeLa cells were grown for 24 hours. The cells were mixed well and placed into an incubator in an atmosphere of 5% CO₂ for 24 hours at 39° C. After a 24-hour killing time, cells were harvested by trypsinization and centrifuged. Cells were resuspended in 100 μl cold PBS with 125 μl 0.4% Trypan Blue subsequently added. Cells were then counted under microscope by phase contrast and fluorescence microscopy.

### Example 3

### High-Throughput In Vitro Cancer-Killing-Activity Assay

To facilitate analysis, a validated high-throughput method of generating CKA among multiple samples was developed. The method involves the use of the RT-CES™ cell electronic sensing system (ACEA Bioscience, Inc.), which measures cellular adherence as a function of electrical resistance. Target cells which are dead or dying lose adherence resulting in decreased resistance, which is detectable in real-time. As described herein, the RT-CES™ platform provided real-time monitoring of tumor cell dynamics as a result of effector function, effector to target cell ratio associations, leukocyte subset functionality, and the stability/seasonality of leukocyte function.

In accordance with carrying out CKA assay in a high-throughput format, desired wells of a RT-CES™ 96-well plate were loaded with 50 μL IVK Medium and blanked according to the manufacturer's instructions. Effector ratios to be used and any additives per well were noted.

Target Cells were maintained through proper culture practice. Live cells were trypsin-harvested and resuspended in IVK Media (50,000/mL) immediately prior to seeding of RT-CES™ 96-well plate. Twenty-four hours prior to the addition of effector cells, 100 μL of a 50,000 target cell/mL suspension was seeded into each pre-determined well, resulting in 5,000 target cells per well in 150 μL total volume. The seeded RT-CES™ 96-well plate was covered and loaded into a 96-well E-Plate Station in a 37° C. incubator, supplemented with 8% CO₂. The plate was scanned for connectivity according to ACEA RT-CES™ Analyzer Instructions. Target cells were allowed to rest, while being actively recorded by RT-CES™ analyzer for up to 24 hours.

Effector cells were obtained by collecting whole blood by venipuncture into 10 mL BD Vacutainer™ Sodium Heparin vials. Blood was transferred to a new 50 mL conical tube containing an equal volume of room temperature 3% Dextran in 0.9% NaCl. The total volume of whole blood used (WBU) was noted. After gently inverting, the solution was left to set at room temperature for 25 minutes. After observing red blood cell sedimentation, the supernatant was transferred to a new 50 mL conical tube. The cells were centrifuged at 250×g for 10 minutes at room temperature. The supernatant was aspirated and the resulting pellet was resuspended in ⅕ WBU. To a new 15 mL conical tube was added Ficoll®-Hypaque (density 1.077) at 1/10 volume of WBU. Subsequently, the resuspended pellet was gently overlaid on the Ficoll®-Hypaque. The tube was centrifuged at 400×g for 30 minutes at room temperature. The agranulocyte fraction was visible as a band among supernatant. This fraction was collected and transferred to a new 15 mL conical tube and diluted with PBS. The pellet, containing granulocytes, was resuspended in 7.5 mL 4° C. 0.2% NaCl and vortexed for 30 seconds. An equal volume of 4° C. 0.2% dextrose in 1.6% NaCl was immediately added and the solution was mixed by gently inverting the tube. Both the agranulocyte and granulocyte fractions were concurrently centrifuged at 250×g for 10 minutes at 4° C. The supernatant was aspirated and the pellet was resuspended in 5 mL IVK Media. Cell number/volume was determined by Trypan blue exclusion principle and the cells were resuspended in IVK Media at a concentration reflective of desired effector to target ratio using the following equation:

2XY=number of effector cells used per well,

wherein X is the target cell seeding number (Target Cells must have doubling time of 24 hours) and Y is the quotient of desired effector number divided by target number at time of addition.

To monitor cell killing activity, RT-CES™ recording of target cells was stopped and the plate was removed from the analyzer. The total volume (150 μL) of each well was manually aspirated without directly touching the bottom of the well. The effector cell suspension was immediately added to well(s) in a 200 μL total volume. An equivalent volume of IVK media was added to control target wells. The active RT-CES™ 96-well plate was covered and loaded into the 96-well E-Plate Station within a 39° C. incubator, supplemented with 8% CO₂. The plate was scanned for connectivity according to ACEA RT-CES™ Analyzer Instructions. Cell Index was recorded at time increments of once every 10 minutes for the first 2 hours and once every 30 minutes following. Data was recorded up to 72 hours. After 72 hours, the plate was removed and collected data analyzed for cancer killing activity by comparing the recorded cellular index of control target wells to experimental wells at each time point taken.

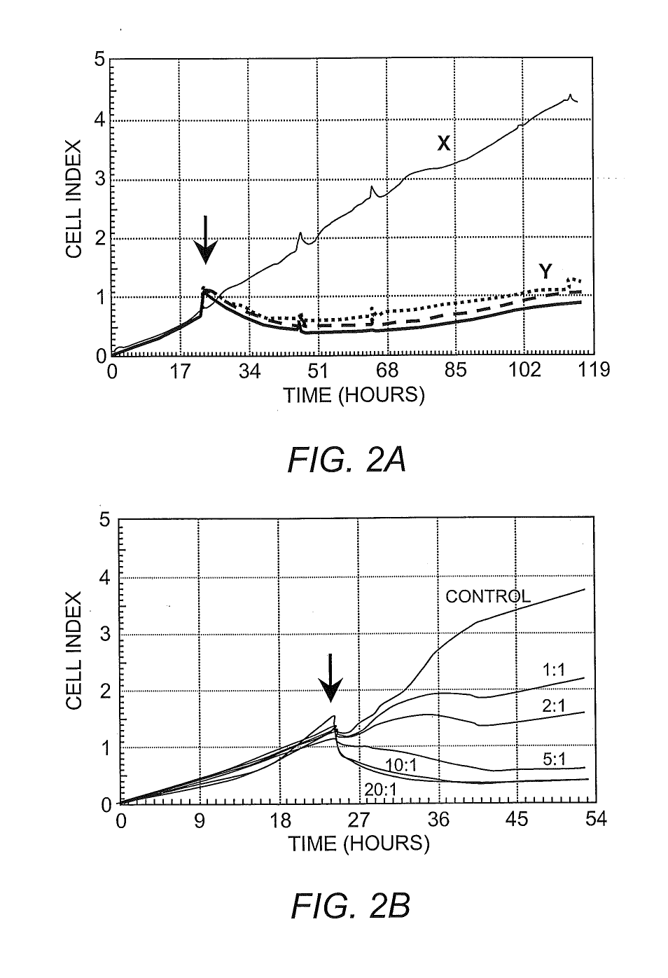

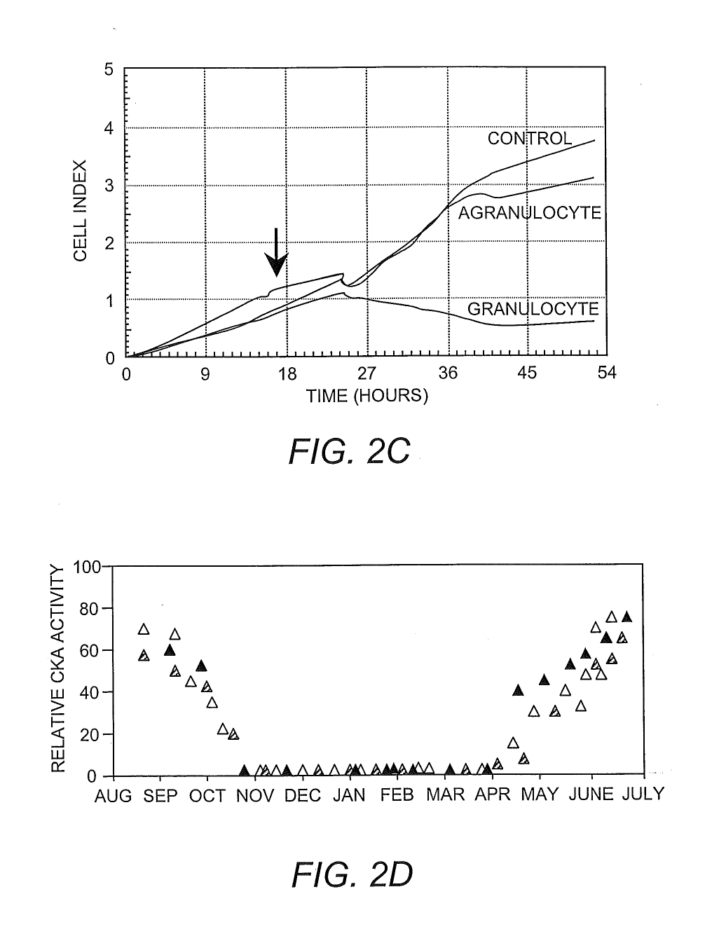

Analysis of cancer-killing-activity of effector cell populations from three individual human subjects indicated that cell death and decreased adherence of cancer cells was mediated by effector cells (FIG. 2A). Furthermore, this CKA was dose-dependent, as increased effector dose resulted in higher CKA and therefore less target adherence (FIG. 2B). Separation of white blood cells into granulocyte and agranulocyte types indicated that the CKA was present in the granulocyte fraction (FIG. 2C). Moreover, CKA was observed to be a seasonal phenomenon as CKA dropped during the winter months (FIG. 2D).

### Example 3

### Granulocytes as Effector Cells

Given that in vitro CKA for WBCs was attributed to the granulocyte fraction, the in vivo CKA of granulocytes is expected to be useful in the treatment of cancer. It is contemplated that granulocytes migrate toward and kill malignant cells. Thus, it is contemplated that either granulocytes pheresis or granulocytes/platelets pheresis from selected individuals will passively transfer anti-cancer activity to the patient in a dose-dependent manner.

Granulocyte concentrates are typically collected by a hemapheresis technique. Granulocyte pheresis usually contains many other leukocytes and platelets as well as 20-50 mL of red cells. The number of granulocytes in each concentrate is ≧1.0×10¹⁰. Various modalities can be used to improve granulocyte harvest, including donor administration of granulocyte colony-stimulating factor and/or corticosteroids (Price, et al. (2000) Blood 95:3302-3309). The final volume of the granulocyte pheresis product is 200-300 mL including anticoagulant and plasma. Red cell sedimenting agents, such as hydroxyethyl starch (HES), are typically used in the collection of granulocytes. Desirably, granulocyte pheresis is administered as soon after collection as possible due to well-documented deterioration of granulocyte function on short-term storage.

Granulocytes pheresis is used conventionally in the treatment of neutropenic patients (generally less than 0.5×10⁹/L [500/μL]) in whom eventual marrow recovery is expected, who have documented infections (especially gram-negative bacteria and fungi), and who have not responded to antibiotics. Granulocytes are administered via a standard blood infusion set because depth-type microaggregate filters and leukocyte reduction filters remove granulocytes. Once granulocyte transfusion therapy is initiated, support should continue at least daily until therapy is completed or the physician in charge decides to halt the therapy. A total cell dose of 2×10¹¹/day is consistent with the current published dosing regimens and with the Circular of Information ((July 2002) Prepared jointly by: American Association of Blood Banks, America's Blood Centers, American Red Cross).

Transfusion of preselected granulocytes that contain the vast majority of CKA into cancer patients will provide a means for selectively killing cancer cells and lesions without harming normal cells. Indeed, in preclinical testing, treatments using white blood cells from cancer resistant donors have completely cured lethal sarcoma, leukemia and prostate cancers in mice. These types of mouse cancer have never been treated successfully by any existing cancer therapy. Thus, the instant method can bring a much better efficacy than conventional cancer therapies. Also, because the therapeutic agents of the present invention are granulocytes that are present to protect healthy humans, minimal adverse side effects are expected.

The foregoing is illustrative of the present invention, and is not to be construed as limiting thereof. The invention is defined by the following claims, with equivalents of the claims to be included therein.

1-9. (canceled)

10. A pharmaceutical formulation comprising white blood cells in a pharmaceutically acceptable carrier, wherein: (i) said white blood cells comprise innate immunity cells selected from the group consisting of natural killer cells, polymorphonuclear leukocytes, monocyte/macrophages, and combinations thereof; (ii) said innate immunity cells are preselected in vitro to kill cancer cells in vitro; and (iii) said white blood cells comprise less than 10% by number of cytotoxic T lymphocytes.

11. The formulation of claim 10, wherein said white blood cells are in vitro cultured white blood cells.

12. The formulation of claim 10 in injectible form.

13. The formulation of claim 10 in unit dosage form and containing from 10⁶ to 10¹⁴ of said white blood cells.

14. The formulation of claim 10 wherein said white blood cells are human cells.

15. The formulation of claim 10, wherein said white blood cells are dog cells.

16. A method of screening human or dog innate immune cells in vitro for cancer killing activity, comprising:
(a) providing white blood cells comprising innate immune cells; and then
(b) contacting said white blood cells to cancer cells in vitro for a period of time; and then
(c) detecting whether or not said innate immune cells kill said cancer cells.

17. The method of claim 16, wherein said contacting step is carried out at a temperature of 35 to 42° C. for a time of 6 hours to 6 days.

18. The method of claim 16, wherein said cancer cells are selected from the group consisting of lung, colon, liver, prostate, ovarian, breast, thyroid, bone, brain, kidney, skin, leukemia cancer cells, lymphoma cancer cells, and combinations thereof.

19. The method of claim 16, wherein said contacting step is carried out by contacting said white blood cells to a plurality of (a “panel”) of different cancer cells.

20. The method of claim 16, wherein said detecting step is carried out by cell electronic sensing.

21-22. (canceled)
